(19) United States
(12) Patent Application Publication
(10) Pub. No.: US 2020/0342659 A1
(43) Pub. Date: Oct. 29, 2020

(54) Structure that Correlates and Separates with Rotation of a Three-Dimensional Element

(71) Applicant: Christine Marie Kennefick, Reston, VA (US)

(72) Inventor: Christine Marie Kennefick, Reston, VA (US)

(21) Appl. No.: 16/750,249

(22) Filed: Jan. 23, 2020

Related U.S. Application Data
(60) Provisional application No. 62/837,239, filed on Apr. 23, 2019.

(51) Int. Cl. G06T 17/00; G16B 45/00

(52) U.S. Cl. CPC G06T 17/00; G16B 45/00

(57) ABSTRACT

A structure with a model comprising a three-dimensional element is provided that in one embodiment maps data, and in a second embodiment dissolves, shatters, breaks or ruptures a disease cell membrane. Vertices, faces and the center of a three-dimensional element provide locations for knots and strands in a model and also provide linkage points for line segments that connect knots and strands. The line segments can fold a structure at linkage points to create a new three-dimensional element or to change a bond angle. Knots and strands can interact with strain components that arise from rotation of the three-dimensional element in a shearable medium. Correlation and separation of paths of strands under rotational strain can further map data, or in a model can promote dissolution of a disease cell membrane.

### FIELD OF THE INVENTION

The present invention relates to both data and microscopic structures that promote the formation, correlation, separation, fracture, dissolution, or rearrangement of other data and microscopic structures in the fields of data analytics, electronics, medicine, chemistry, biology and physical mathematics.

### BACKGROUND OF THE INVENTION

There is a need not only to store and retrieve data rapidly, but to provide structures onto which data can be mapped so that data can be correlated and separated quickly as a situation indicates. In one embodiment, this invention provides a first mapping of data onto strand like and knot structures that can change both their shape and interaction with one another. This invention comprises a second mapping in which structures have a model comprised of knots and strands that in turn are mapped onto line segments. The line segments link at linkage points to create a three-dimensional element with its own symmetry and orientation. The line segments can also fold at linkage points to change an angle between the line segments. The result of the folding can be a new three-dimensional element or a change in symmetry in an original element. Any one or more of vertices, face center of masses and center of the three-dimensional element provides a linkage point of the line segments. Rotation of the three-dimensional element provides a set of rotational strains that can be used to change the shape and interaction of the knots and strands.

It is also a current challenge that therapies presently available for treating cancer and other diseases might be difficult for a patient to tolerate physically, might be expensive and not easily accessible, and might not eliminate a necessary number of cancer cells in a patient. A second purpose of the invention here is to fill a gap in a medical need for a cancer or other disease therapeutic that is easily tolerated, accessible, relatively inexpensive, and one that can eliminate cancer or other disease cells in both early and late stages when other therapies have unfortunately not eradicated a necessary number of disease cells.

This invention therefore seeks in a second embodiment to offer a structure that mechanically, selectively and reliably breaks a disease cell membrane by a release of strain energy to incapacitate the cell. One challenge to this approach is that the outer membrane of a cancer cell, for example, is very pliable and becomes even more so when it is strained. Hence attempting to break a cancer cell membrane by having a molecule poke or burrow through such a membrane might simply leave the cell intact. This second embodiment is comprised of electronic, atomic and molecular structures that in a model develop a pattern of strain energy selectively in a disease cell membrane, but not in normal cells. A release this this strain energy dissolves, shatters or ruptures the membrane to eliminate the disease cell.

### BRIEF SUMMARY OF THE INVENTION

The present invention is a structure with a model that comprises a plurality of strand like and knot structures upon which data can be mapped. The plurality of strand like and knot structures can be both represented and linked together at linkage points by line segments. The line segments can form a large three-dimensional element with planar faces that has a different or same symmetry as structures formed by a linkage of the strand like structures. Vertices, face center of masses and the center of a three-dimensional element can provide linkage points of the line segments onto which the strand like and knot structures are mapped. It is an object of the three-dimensional structure to rotate and provide strain that will cause the original strand like structures to move in one direction together or to separate. In a first embodiment, a mapping of data onto a three-dimensional element, its knots and strands, and rotational strain cause the original data to correlate or separate under distinct conditions that are yet changeable by a user of the mapping.

In second embodiment, the present invention is a microscopic structure that utilizes features of a cancer cell that differentiate it from normal cells to cause a lipid bilayer in a cancer cell membrane to breakup or dissolve. It is one object of this invention to use a rapid uptake of glucose of a cancer cell to work against the cell in that a large potential energy gradient needed for faster diffusion may facilitate a breakup of a membrane of the cell. It is another object of the present invention to utilize a lack of recognition between one cancer cell and other cells around it by providing a structure forms laterally across a cancer cell membrane to help dissolve it. It is yet one more object that a microscopic structure in this invention is comprised of glucose or another carbohydrate molecule that is potentially tolerable, affordable and easily accessible to a patient.

### BRIEF DESCRIPTION OF THE DRAWINGS

Reference will be made to the accompanying drawings, which can represent any length scale from atomic to continuum levels.

FIG. 1 shows an overview line segments that link at linkage points and that fold a structure to produce a face of a three-dimensional element with a threefold axis of symmetry.

FIG. 2 shows both a structure that folds and outlines of structures that can further encapsulate a structure that folds.

FIG. 3 shows a rotation of a three-dimensional element to promote formation of a third structure that breaks two structure sets apart.

FIG. 4 shows a schematic overview of a three-dimensional element that undergoes shear strain as strand like parts in the element undergo a change in angular momentum;

FIG. 5 shows a magnified view of a release of strain energy that comprises a rotation of strand like structures into a region of lower curvature;

FIG. 6 shows a model of strain comprising strands that move into a region that is narrower and more highly curved than their original region;

FIG. 7 shows a separation of strand like entities after their paths move toward each other and constrain the strands in too small a space;

FIG. 8 is a model of micelles in a membrane bilayer, regions of strain, and parts of molecular structures causing the strain;

FIG. 9 shows structures that fold to form a three-dimensional element that through rotation can provide strain to drive micelles in a lipid bilayer apart;

### DETAILED DESCRIPTION

The present invention will now be described with reference to the accompanying drawings. In this invention, data can be numerical from a machine or device. The data can also characterize physical phenomena at a microscopic level, such as nucleons, atoms, bonds, molecules and material grains. The data is not limited to numerical values. The data can be incoming, one data element at a time. Alternatively, an entire set of data can be stored in an array or other container and then mapped onto one or more structures in this invention.

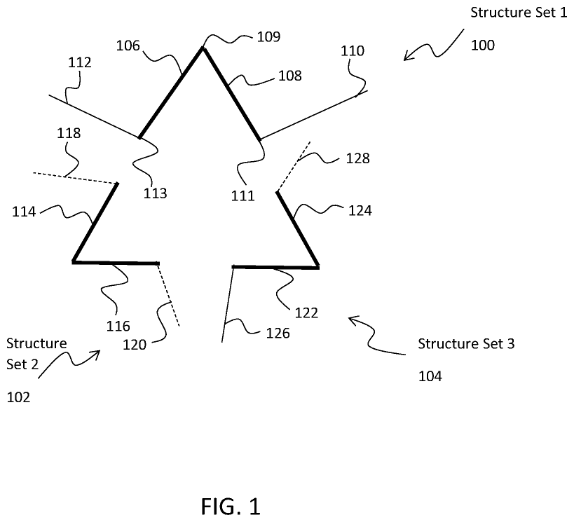

FIG. 1 shows a face of a 3D element that has an n-fold axis of symmetry. In the example in FIG. 1, the face is outlined by an equilateral triangle comprised of three structure sets 100, 102 and 104. Structure set 1, denoted with label 100 in FIG. 1, comprises line segments 106, 108, 110 and 112, some of which are joined end-to-end at linkage points to produce angles between one another. In the example in FIG. 1, the angle between segments 106 and 108 is 60 degrees.

In one embodiment of the invention, data is mapped onto structure set 100 in which segments 106, 108, 110 and 112 and the angles between them represent characteristics of the data set. Segments 106 and 108 are connected at linkage point 109, which allows structure set 100 to fold into various angles between segments 106 and 108. Segments 108 and 110 are connected at linkage point 111, which allows structure set 100 to fold into various angles between segments 108 and 110. Segments 106 and 112 are connected at linkage point 113, which allows structure set 100 to fold into various angles between segments 106 and 112.

Similarly, a second set of data can be mapped onto structure set 2, denoted with label 102 in FIG. 1. Structure set 2 comprises line segments 114, 116, 118 and 120, with an angle of 60 degrees between segments 114 and 116 in the example in FIG. 1. Yet a third set of data can be mapped onto a structure set 3, which in the example in FIG. 1 is element 104, and comprises line segments 122, 124, 126 and 128. The angles between line segments 122, 124, 126 and 128 can be the same or different from the angles between the line segments that comprise structure sets 100 and 102. Correspondingly, the characteristics of the data that are mapped onto structure sets 100, 102 and 104 can be the same or different. Same or different characteristics of the data that are mapped onto structure sets 100, 102 and 104 can also be mapped onto the orientation in space of each of structure sets 100, 102 and 104 in its entirety.

In FIG. 1, three 60-degree angles formed by line segments 106, 108, 114, 116, 122 and 124 outline an equilateral triangle that can denote yet a further characteristic common to all three data sets that are mapped onto structure sets 100, 102 and 104. In another embodiment, one single data set can be mapped onto all three structure sets 100, 102 and 104 as a whole. In such a mapping, characteristics of said single data set mapped onto orientation angles of structure sets 100, 102 and 104. A characteristic of a single data set can also be mapped onto the equilateral triangle formed by all three structure sets.

In a second embodiment of this invention, a three-dimensional element in FIG. 1 outlines molecules that can promote strain. Outlining a molecule means that the three-dimensional element has planar faces and that atoms, nucleons or electrons of the same or different molecules may be at any one or more of vertices, face center of masses and the center of the three-dimensional element. A face itself can have edges formed by bonds of a plurality of molecules. In a model, any one or more of nucleons, atoms, electrons, bonds and molecules are mapped onto knots and strands. Line segments link the strands and knots. The line segments link together and fold a structure at linkage points to change an angle between the line segments. Any one or more of vertices, face center of masses and centers of the three-dimensional element provides a linkage point of the line segments. In FIG. 1, in this second embodiment, structure sets 1, 2 and 3 become molecule 1, molecule 2 and molecule 3. In FIG. 1, a face of a 3D element outlined by an equilateral triangle, for example, is comprised of bond angles from molecule 1 numbered 100, molecule 2 numbered 102, and molecule 3 numbered 104. Molecules 100, 102 and 104 can be the same or different from each other in atomic elements or in conformations.

In this second embodiment, in FIG. 1, bonds 106 and 108 make an angle of 60 degrees with each other. The 60 degrees is an example: other embodiments of this invention can comprise other bond angles that form another n-fold axis of symmetry, or simply a mirror plane. The connection of bond 108 to bond 110 results in a bond angle that can be the same or different from a bond angle made from a connection of bond 106 with bond 112. Similarly, bonds 114 and 116 make an angle of 60 degrees with each other. A bond angle from a pair of bonds 118 and 114 can be the same or different from a bond angle formed by bonds 116 and 120. Likewise, bonds 122 and 124 form an angle of 60 degrees to complete an outline of an equilateral triangle. The bond angles made from adjacent bonds 122 and 126 and from adjacent bonds 124 and 128 can be the same or different from one another.

In FIG. 1, either for a data mapping or for a representation of a plurality of molecules, the face outlined by the equilateral triangle can be part of an element that comprises only an axis of 3-fold symmetry. In other embodiments, the face can be part of an element such as a tetrahedron or other element with cubic symmetry that comprises four such triangular faces. A face outlined by an equilateral triangle might also be enclosed by a larger face outlined by a 120-degree angle that is part of a right rhombic prism.

In one embodiment of this invention, various forms of glucose or other carbohydrate molecules densify to form a chemical potential gradient that results in a faster diffusion or uptake through a cancer cell membrane. In this density of molecules that is higher than that of normal cells, faces of 3D elements form, and even faces within faces can form, from various bond angles of carbohydrate molecules. Such faces can contain symmetry, as shown in the example in FIG. 1, or alternatively, faces can form a 3D element with different symmetry or no symmetry.

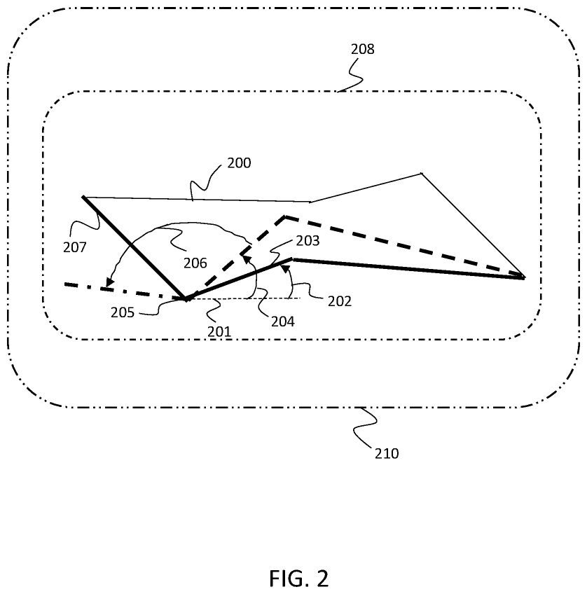

FIG. 2 shows another structure that can provide a mapping of data as the data changes. Data is mapped onto line segments joined end-to-end at linkage points to form a structure 200. As incoming or existent data changes, a line segment 203 can move upward from reference segment 201 to fold structure 200 at linkage point 205 and to change angle 202 to angle 204. Other changes in the data can move a line segment 207 downward at linkage point 205 to produce angle 206 that serves as a label for a new set of data. Additionally, encapsulating structures 208 and 210 can change their size and shape to denote further changes in the data. Such changes in angles between line segments, and the size and shape of an encapsulating structure are such that their original configuration is known to map back to a source that changed the data.

In a second embodiment, FIG. 2 is a model comprising line segments onto which atoms and bonds of a therapeutic glucose or other carbohydrate molecule can be mapped. A structure having the model is comprised of a glucose molecule that approaches and interacts with a cancer or other disease cell membrane. In the model, atoms and bonds are first mapped onto knots and strands which are connected by line segments 203 and 207. Line segments 203 and 207 link together and fold a structure at linkage point 205 to change an angles 202, 204 and 206 between said line segments. In the structure itself, a portion 200 of said glucose or other carbohydrate molecule is comprised of an initial bond angle 202 that can deform or fold molecule 200 at linkage point 205 into another bond angle 204. Bond angle 204 by itself can promote symmetry in a 3D element that spans other molecules. Rotation of a 3D element, either separately or together with a change in bond angle 204, can produce strain, precipitation or a hydrophobic reaction to move micelles in a cancer or other disease cell membrane apart. Alternatively, bond angle 204 can be adjacent to another bond angle 206 that likewise can promote symmetry in a 3D element that can span other molecules. In still another embodiment, a bond angle that is a sum of bond angle 204 and bond angle 206 can produce strain, precipitation or a hydrophobic reaction to move micelles in a disease cell membrane apart.

Portion 200 or an entirety of said glucose or other carbohydrate molecule in FIG. 2 can be encapsulated within another molecule 208, which in turn can be encapsulated within yet another molecule 210. Molecule 208 can be a carrier molecule that aids diffusion of a glucose or other nutrient molecule through the cancer cell membrane. Carrier molecule 208 can already be in the membrane when the glucose or other nutrient molecule arrives, or molecule 208 can arrive at the membrane along with said glucose or another nutrient molecule. Molecule 210 can be an encapsulation molecule that aids transport of said therapeutic glucose or other carbohydrate molecule to a cancer or other disease cell membrane. Both molecules 208 and 210 may or may not be part of promoting strain that drives micelles in a cancer or other disease cell membrane apart.

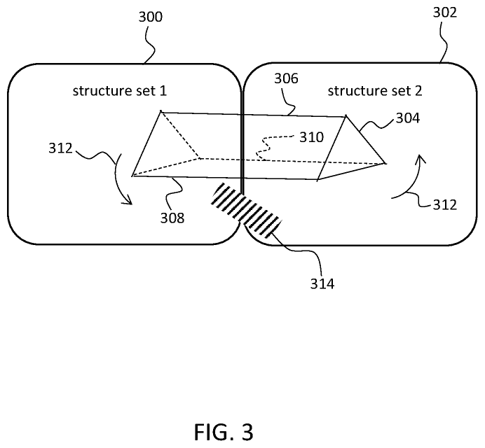

FIG. 3 shows structure set 1, labeled as element 300, and structure set 2, labeled as element 302. Structure set 1 has a model comprised of line segments that form one face of a three-dimensional element 304 that has planar faces. Similarly, structure set 2 has a model comprised of line segments that form another face of the three-dimensional element 304. Any one or more of vertices, faces and center of the three-dimensional element provides a linkage point of the line segments.

In a first embodiment, the line segments that form the faces of three-dimensional element 304 are the result of data being mapped onto the line segments, with data being from a machine or device, or data that characterizes nucleons, atoms, atomic clusters or grains of a material. Data is not limited to these examples, but can be any data that is mapped for convenience onto the line segments. In a model, line segments can link knots and strands that are located at or extend from the center, vertices or face center of masses of element 304. Although element 304 in FIG. 3 is shown with end faces outlined by triangles, element 304 can have any symmetry or no symmetry at all. Line segments 306, 308 and 310 that connect the end faces of element 304 can be the result of data that has been directly mapped onto them. Alternatively, the line segments 306, 308 and 310 that span across structure sets 1 and 2 and that form the three dimensional element 304 can be the result of an interrelationship or lowering of energy between quantities that have been mapped onto the line segments that outline the end faces of element 304.

In a model, element 304 in FIG. 3 undergoes a rotation through an angle 312 within a shearable medium. The rotation results in rotational strain components that arise from summing of strains over a nearest face of element 304. In a first embodiment, the rotational strain results in element 314 that is a structure that further characterizes or connects data structures 1 and 2.

In a second embodiment, the two structure sets in FIG. 3 are two cancer or other disease cells. Schematically, FIG. 3 shows a breaking of a cancer or other disease cell membrane in a model by a formation of a precipitate that spans membranes of two cancer or other disease cells. In FIG. 3, structure set 1, labeled element 300, represents in a model a membrane of cancer cell 1. Structure set 2, labeled element 302, represents a membrane of cancer cell 2. In yet models of other embodiments, 300 and 302 represent membranes might enclose inner parts of one or both cells.

A three-dimensional element 304 spans across membrane 300 and membrane 302. At vertices, at center-of-mass of faces, or at the center of element 304, are strands and knots upon which atoms, electrons or atomic clusters have been mapped. Any one or more of vertices, faces and center of the three-dimensional element 304 can provide a linkage point of a line segment that connects any one or more of the knots and strands. Here in this model, the interaction is not one to cause separation, but one to cause bonding or correlation. Such bonding or correlation is modeled by strands, that when sampled in a model, have paths that go in the same direction.

Such correlation may be preceded or followed by any number of rotations through a variable angle 312 of three-dimensional element 304 that produce rotational strain components that interact with and affect the shapes of the strands. Rotation through a variable angle 312 may also promote symmetry within element 304 or any other three-dimensional element adjacent or close to element 304. Rotations through an angle 312 may be about axes of any orientation and about axes with pivot points on or within element 304. Said correlation of strands around or within element 304 promotes a precipitation 314 that drives micelles of cancer or other disease cells apart to dissolve, shatter or rupture membranes within or enclosing each of the two cells.

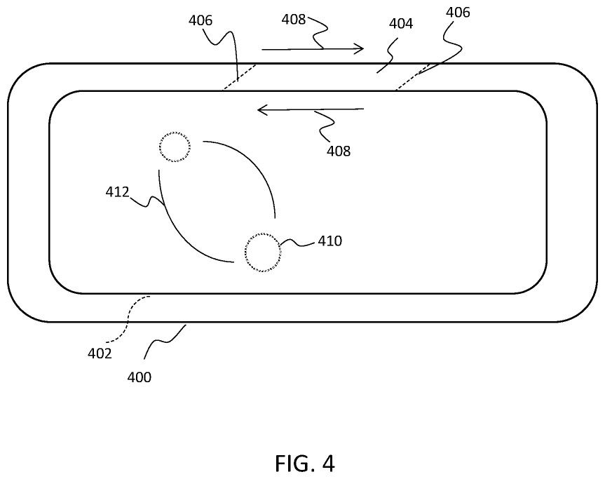

FIG. 4 shows an outer boundary 400 and an inner boundary 402 of a structure onto which data can be mapped in a model. In a first embodiment, data is mapped onto the structure with boundaries 400 and 402. Element 404 is a face with edges 406 that belong to a three-dimensional element in a model within a structure with boundaries 400 and 402. Data can be mapped onto a three-dimensional element with face 404, and also upon yet a third structure 410. Face 404 undergoes a shear or other strain that interacts with the data so as to change the shape of or correlation between the original structures onto which the data was mapped. The shear or other strain can arise from a structure 410 that undergoes a change in orientation or momentum 412.

In a second embodiment, FIG. 4 shows an overview of a mechanical interaction in a model that leads to a pattern of strain energy within a cancer or other disease cell membrane. The solid lines 400 and 402 schematically depict outer and inner boundaries, respectively, of a cancer or other disease cell membrane. Similarly, 404 is a view of a face with edges 406 of a 3D element within a model inside a membrane with boundaries 400 and 402. The three-dimensional element in a model can have atomic, electronic or potential energy configurations that map onto knots and strands at any one or more of its center, vertices and face center of masses. The atomic, electronic or potential configurations 410 may be within or outside said three-dimensional element with face 404.

In FIG. 4, in this second embodiment, face 404 of a three-dimensional element in a model goes a shear strain 408 that can be caused by electronic or atomic configurations 410 that undergo a change in direction or angular momentum 412. FIG. 4 shows but one example in this invention of a strain that can lead to a strain energy pattern within a cancer or other disease cell membrane. In other embodiments, shear strain 408 can be another strain that is tensile, compressive, or a shear across another plane. In other embodiments, the cause of the strain in the model and rotation of the three-dimensional element can arise from forces coming from outside a cancer or other disease cell, or from an interaction with a molecule approaching a cancer or other disease cell.

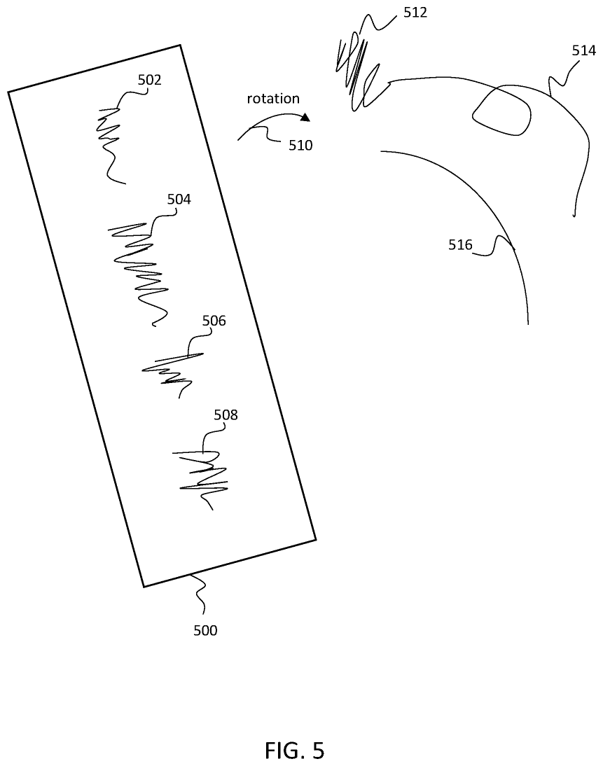

In a first embodiment, FIG. 5 shows an overhead planar view of a three-dimensional element 500. In a model, data has been mapped onto strand like structures 502, 504, 506 and 508. Although structures 502, 504, 506 and 508 are shown as loose strands, they can be more tightly wound into knots. Strain from a rotation 510 of element 500 results in a change of shape in any one or more of structures 502, 504, 506 and 508. An example shape 512 transforms under the strain into a shape 514 that has fewer loops per unit volume. Shape 514 then maps data over a larger radius of curvature 516.

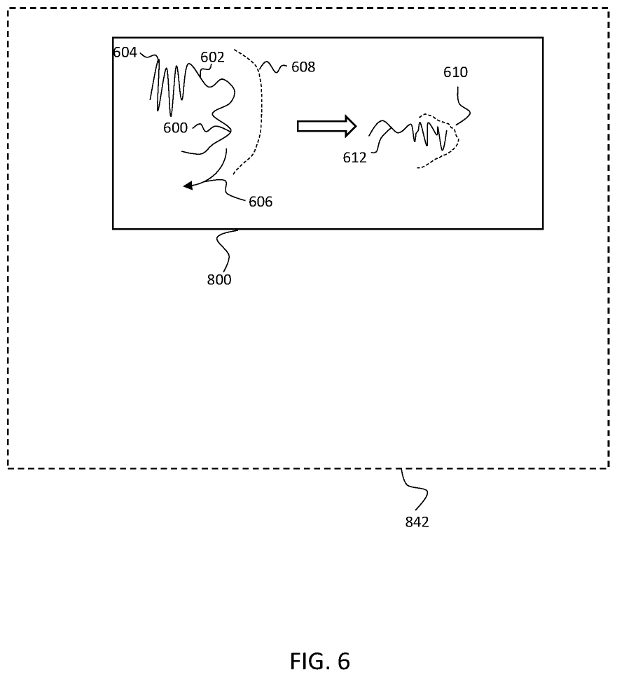

In a second embodiment, FIG. 5 shows detail of a release of a pattern of strain energy that is one definition of a release of energy in this invention. A region 500 is comprised of one or a plurality of electronic configurations or energy envelopes 502, 504, 506 and 508. An energy envelope is a succession of points in space, each corresponding to an amount of energy. The succession of points becomes a boundary to or a contour representing other energy values at points nearby. In a model, strands 502, 504, 506 and 508 have a relatively large number of loops in a small radius of curvature, similar to strand 612 in FIG. 6. A rotation 510 of a region 500 allows a larger density of loops in strands representing an electronic configuration 512 to release a pattern of strain energy. Configuration 512 spreads out into a smaller density of loops 514 along a larger radius of curvature 516. Such a release of strain energy in a model in this second embodiment would dissolve, break, shatter or dissolve a disease cell membrane.

Although FIG. 5 shows in a model a release of strands confined within a unit volume, a structure having a model in this invention can be comprised of a release of strain energy that is defined in other terms. The release of strain energy might involve, for example, a stress concentration that decreases to a value smaller than what it originally had.

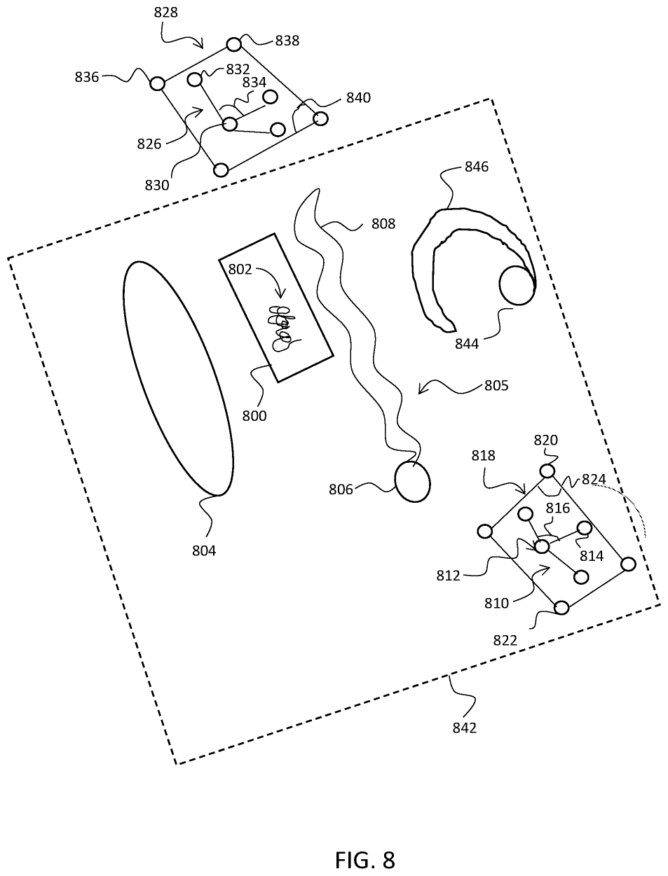

FIG. 6 shows more detail on a mechanism that is one definition strain in this invention. FIG. 6, for example, shows a model of buildup of strain in a region 800 shown in FIG. 8 before any strain is released within a broader region 842. In FIG. 6, a strand 600 is comprised of inflection points 602, which are points of transition between negative and positive curvature, or between positive and negative curvature. Strand 600 is also comprised of loops 604, which are curved turning points between inflection points 602. In a model, strand 600 can be, for example, an electronic configuration, or an envelope that is a succession of points in space having different values of potential energy. In another embodiment, strand 600 can also be a structure upon which data is mapped.

A rotation 606 of strand 600 along a relatively large radius 608 can allow a reduction in strain, as evidenced by a reduction in a number of loops or inflection points in a strand 600 representing an electronic configuration, energy envelope or data mapping. If strain increases, a radius of rotation 610 becomes smaller. Strand 600 then becomes represented by strand 612, wherein a number of loops and inflection points of a strand 612 increases within a smaller radius of rotation 610.

Although FIG. 6 shows a displacement of strands into a smaller unit volume, a structure with a model in this invention can be comprised of strain defined with other terms. Other terms can include but are not limited to displacements per unit length, infinitesimal displacements per unit length, and displacements averaged over a length of an atomic cluster or over a length dimension of a three-dimensional element that spans a plurality of clusters. A strain can also be the result of a contact stress, and of a summation over a face of a three-dimensional element causing a contact stress.

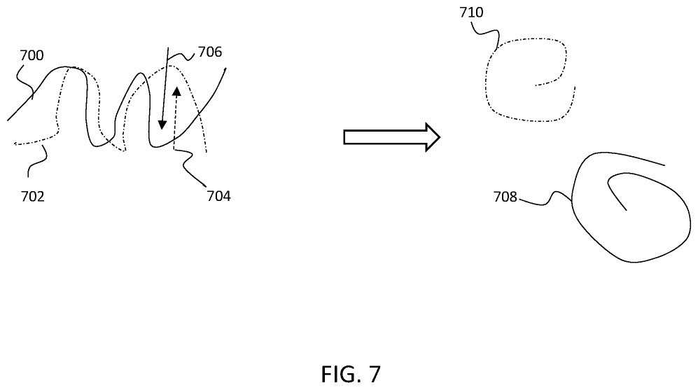

FIG. 7 shows two strands 700 and 702 in this invention that are oscillating up and down and interacting with each other. In a model, strands 700 and 702 can represent wavelike nature of electrons or atoms, or simply an envelope that is comprised of successive values of kinetic or potential energy. In one embodiment, data is mapped onto strands 700 and 702, which represent an interaction between data sets. In a second embodiment, the strands can interact within one micelle or across two different micelles in a disease cell membrane. During an interaction, if a path 704 of strand 700 moves upward and a path 706 of strand 702 moves downward, the two strands might attempt to place too much energy within a single volume of space. The result is that the two strands separate into a configuration in which strands 700 and 702 have shapes 708 and 710, respectively. In one embodiment, data that is mapped onto the two strands separate into two data sets. In second embodiment, such a change in shape of a plurality of strands becomes in a model a separation of micelles, or a break in a cancer or other disease cell membrane.

FIG. 8 shows an overview of components that comprise an embodiment to break up a disease cell membrane. In a model, a region of strain 800 comprises a strand or knot like electronic configuration 802 whose density displaces and increases as it occupies a volume less that it normally would. In this embodiment, a strand is a shape comprising loops and inflection points onto which electron and atomic configurations have been mapped. A strand might also represent any one or more of successive values in potential energy, a wavelike nature of an electron, an atomic nucleus, a nucleon, an atomic bond, an electromagnetic wave, a strain wave and a sound wave.

A structure within a schematic region 804 can cause a strain 800. Strain can comprise fissures to produce a stress concentration, for example. Or strain 800 can be the result of a molecule that moves either a hydrophilic head 806 or a hydrophobic tail 808 of a micelle 805 in a cancer or other disease cell membrane.

Alternatively, in the vicinity of a micelle 805, there can be an atomic cluster or molecule 810 that comprises one type of atom 812, further comprises another type of atom 814, with a bond angle 816 between said atoms that either stays constant or can change as a molecule folds itself. In a model, atomic cluster or molecule 810, atom 812, atom 814 and bond angle 816 can be mapped onto strands and knots and onto line segments that link together at linkage points, as illustrated in FIG. 1 and FIG. 2. Hence bond angles that either stay constant or can change as a molecule folds itself comprise a model wherein line segments can link strands and knots and wherein line segments link together and can fold at linkage points to change an angle between the line segments, as shown in FIGS. 1 and 2. In a preferred embodiment, atomic cluster 810 is a form of glucose that is easily tolerable to a patient. In this embodiment glucose forms potential energy gradients that are higher than normal to accommodate a rapid uptake of glucose that differentiates a cancer or other disease cell from that of a normal cell.

Another microscopic structure 818 can encapsulate cluster 810. The encapsulating structure 818 comprises an atom 820, can further comprise a different atom 822, and can have a variable bond angle 824 that can change as it interacts with cluster 810, hydrophilic head 806 or hydrophilic tail 808 of a micelle 805. In a model, atoms, nuclei and electrons of cluster 810 and its encapsulating structure 818 can be mapped onto strands and knots, which can be further mapped onto line segments. The line segments link together at linkage points and can fold at a linkage point to change a bond angle 824.

Atomic cluster 810 with its encapsulating molecule 818 can interact across a micelle 805 with another atomic cluster 826 that has its own encapsulating structure 828. Atomic cluster 826 can be the same or different from cluster 810. Likewise, encapsulating structure 828 can be the same or different from encapsulating molecule 818. Atomic cluster 826 comprises an atom 830, another atom 832 that can be different or the same as atom 830, and a bond angle 834 that can change as cluster 826 interacts with cluster 810 or with micelle 805. Similarly, the encapsulating structure 828 comprises an atom 836, another atom 838 that may be the same or different from atom 836, and a bond angle 840 that can change as the encapsulating molecule 828 interacts with atomic cluster 826 or with any other cluster, with micelle 805 or with any other micelle, or with encapsulating molecule 818 or any other encapsulating molecule.

In a model, atoms, nucleons and electrons of atomic cluster 826 and its encapsulating molecule 828 can be mapped onto knots and strands. The knots and strands can be further mapped onto line segments that link at linkage points. The line segments can link together to create a three-dimensional element with planar faces. The three-dimensional element rotates within a shearable medium to produce strain that interacts with the knots and strands. The line segments can also fold at linkage points to change angles 834 and 840.

In a preferred embodiment, both atomic clusters 810 and 826 are parts of glucose molecules or another molecule that a cancer or other disease cell selectively uptakes faster than normal cells do. Clusters 810 and 826 are parts of structures that can fold or rotate themselves so that one or more bond angles provide symmetry to three dimensional elements that span across micelles in a cancer membrane. The symmetry can promote higher potential energy gradients, diffusion, or even precipitation in directions across as well as through the membrane to break it up either through release of elastic strain energy or through a hydrophobic reaction. Traversing across a cancer cell membrane to a membrane of an adjacent cell, such diffusion or precipitation could be selective by utilizing a lack of recognition of one cancer cell with another in comparison with normal cells.

FIG. 8 depicts a structure having a model in which strain in region 800 is released within a region 842. In a model, strain energy rotates out of regions of confined strain, as shown in FIGS. 5 and 6. In such a release of energy, both a head 844 and a tail 846 of a micelle and the knots, strands, line segments and three dimensional elements upon which they can be mapped can move, rotate, and even form new three dimensional elements, thus breaking or dissolving a cancer or other disease cell membrane.

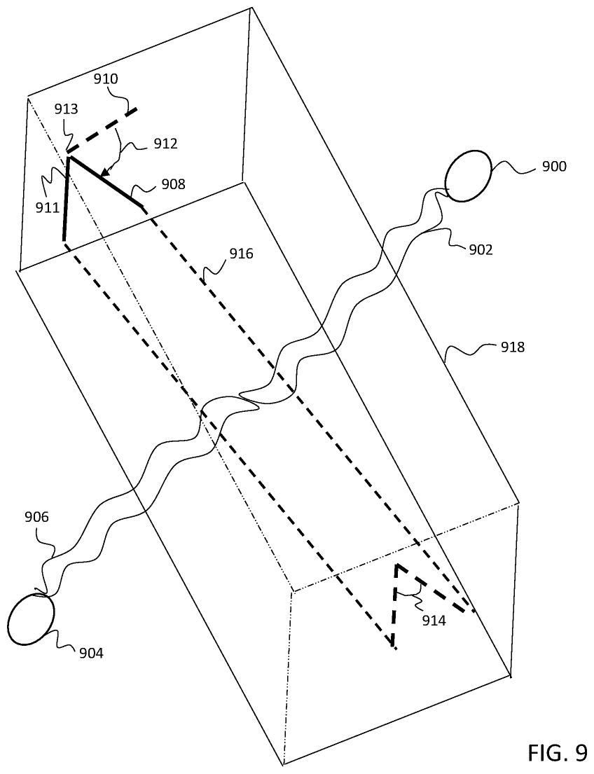

FIG. 9 shows a detailed view and model of symmetry forming between two molecular elements to promote strain or precipitation across micelles in a disease cell membrane. A micelle with hydrophilic head 900 and hydrophobic tail 902 forms a bilayer with another micelle comprised of a hydrophilic head 904 and hydrophobic tail 906. In this model, line segments upon which another molecule is mapped comprise a bond angle 908 that is 60 degrees and produces a threefold axis of symmetry. Bond angle 908 is a result of a line segment 910 being linked to line segment 911 at a linkage point 913 and rotating through an angle 912. A similar bond angle 914 of another molecule exists on an opposite side of two micelles.

In a model in FIG. 9, a three-fold axis and a mirror plane are part of a three-dimensional element 916 that forms. Atomic and electronic configurations are mapped onto knots and strands which are in turn mapped onto vertices, face center of masses and the center of element 916. Line segments can link the knots and strands. Any one or more of vertices, face center of masses and center of element 916 can provide linkage points of the line segments as they connect together in model.

Rotation of three-dimensional element 916 within a shearable medium produces strain components that interact with the shape and direction of the knots and strands in a model. The shape and direction of the knots and strands promote a strain as represented in FIGS. 4, 5 and 6, a hydrophobic reaction represented in FIG. 7, or a precipitation represented in FIG. 3. A release of strain energy from such strain drives the micelles apart. Symmetry in element 916 might promote a lower potential energy for a precipitate, for example, or might promote a region of strain nearby where such symmetry does not exist.

Finally, atoms, nucleons, and atomic clusters of one or more carrier or encapsulation molecules can also be mapped onto knots and strands in a model. The knots and strands can be mapped in turn onto vertices, face center of masses and the center of a three-dimensional element 918. Any one or more of the vertices, face center of masses and center of the three-dimensional element 918 can provide linkage points where the line segments can link together, fold at linkage points to change an angle between the line segments, or even form another three-dimensional element. Rotation of any three-dimensional element formed in these models within a shearable medium can promote a strain, a hydrophobic reaction or a precipitation that drives the micelles apart.

In a preferred mode of this invention, a structure has a model that comprises three-dimensional elements that may exist throughout the structure. The three-dimensional elements may have different symmetries and may have dimensions at different length scales. Symmetry can be comprised of any one or more of a mirror plane, n-fold axis of symmetry, and an inversion center. Nucleons at can be mapped onto vertices, face center of masses or centers of the three-dimensional elements and onto line segments that link at linkage points, wherein said nucleons are subatomic particles inside nuclei of one or more atoms. Atoms of one or more atomic clusters can be mapped onto vertices, face center of masses or centers of said three-dimensional elements and onto line segments that link and fold at linkage points, wherein said atomic clusters are a plurality of atoms joined by bonds and may be part of a solid or may be a part of a molecule. The three-dimensional elements can have molecules in whole or in part at their vertices, faces or centers.

In a preferred mode of this embodiment, breakage of a cancer or other disease cell membrane arises from a plurality of mechanisms that drive micelles in a membrane apart. Examples of these mechanisms are described in more detail here. A glucose or other carbohydrate molecule arrives at a cancer cell membrane, for example, to provide nutrients for the cancer cell. A glucose or other carbohydrate might arrive with its own carrier molecule, which is a molecule that promotes a change of shape and passage of the glucose or other carbohydrate molecule through a cell membrane. Another molecule can serve to encapsulate both the glucose and carrier molecule on its way to the cancer cell itself and can have both hydrophilic and hydrophobic parts. In a preferred mode, the carrier molecule allows the glucose or other carbohydrate molecule to move both toward the cell center and laterally through the membrane. The lateral movement might be from either high potential energy gradients for faster diffusion, or a lack of recognition of one cell to the cell adjacent to it. A higher energy in a gradient for diffusion might manifest itself in a lack of symmetry in a 3D element that spans across two or more glucose molecules.

To form a potential energy gradient, a three-dimensional element extending across two or more glucose molecules in a model might then rotate to provide symmetry elements among atoms to lower energy at another location. In a model, a symmetry element might lower energy by allowing a concentration of loops and strands to rotate out more easily among atoms and molecules, as shown in FIGS. 5, 6 and 7. A combination of any one or more of a high density of molecules, lateral diffusion through hydrophobic tails of the micelles in a membrane, rotation of three-dimensional elements, or an elevated amount of stiffness would provide a strain energy pattern. A release of this strain energy pattern in part or in its entirety by separation of strands or by correlation of strands to form new bonds or to form a precipitate would drive micelles apart in a model. A structure having such a model might also include movement of hydrophobic tails of micelles to hydrophobic parts of an encapsulation molecule.

Although particular embodiments of the invention have been described in detail herein with reference to the accompanying drawings, it is to be understood that the invention is not limited to these precise embodiments, and that various changes and modifications may be effected therein by one skilled in the art without departing from the scope or spirit of the invention as defined by the appended claims.

What is claimed:

1. A structure having a model comprising any one or more of strands and knots onto which data is mapped, with said model comprising
a. line segments that link said strands and knots, and that link together and that can fold a structure at linkage points to change an angle between said line segments;
b. a three-dimensional element, wherein any one or more of vertices, face center-of-masses and center of said three-dimensional element provides a linkage point of said line segments;
c. strain components that arise from a rotation of said three-dimensional element in (b);
d. a correlation or separation of said strands as they interact with strain components in (c).

2. A structure having a model that breaks, dissolves, shatters or ruptures a disease cell membrane, with said structure comprising
a. a glucose molecule;
b. a molecule that encapsulates said glucose molecule;
and with said model comprising
d. strands and knots that onto which any one or more of nucleons, atoms, electrons, bonds and molecules are mapped;
e. line segments that link said strands and knots and that link together and fold a structure at linkage points to change an angle between said line segments;
f. a three-dimensional element, wherein any one or more of vertices, face center of masses and centers of said three dimensional element provides a linkage point of said line segments;
g. strain components that arise from rotation said three-dimensional element in (f);
h. a correlation or separation among said strands as they interact with said strain components in (g).
